(19) United States
(12) Patent Application Publication
(10) Pub. No.: US 2016/0220789 A1
(43) Pub. Date: Aug. 4, 2016

(54) DEVICES, SYSTEMS, AND METHODS FOR SPG BLOCK AND NEURO-MODULATION

(71) Applicant: Stephen Eldredge, South Jordan, UT (US)

(72) Inventor: Stephen Eldredge, South Jordan, UT (US)

(21) Appl. No.: 14/682,596

(22) Filed: Apr. 9, 2015

Related U.S. Application Data
(60) Provisional application No. 61/978,530, filed on Apr. 11, 2014.

(51) Int. Cl. A61M 25/00; A61L 29/16; A61M 5/145

(52) U.S. Cl. CPC A61M 25/0097; A61M 25/0026; A61M 25/0068; A61M 5/1452; A61L 29/16; A61M 2205/586; A61M 2025/0008; A61M 2205/582; A61M 2210/0618; A61L 2300/436; A61L 2300/204

(57) ABSTRACT

Neuro-modulation of the SPG is achieved by direct delivery of medication that includes a CGRP receptor antagonist using a trans-nasal drug delivery device with delivery features that may include an ergonomic grip, directional control and indicators, or radiopaque portions.

### Related Applications

This application claims the benefit of, and priority to, U.S. Provisional Application Ser. No. 61/978,530, filed Apr. 11, 2014, the contents of which are incorporated by reference.

### BACKGROUND

Both acute and chronic headaches have serious impacts upon the ability of people to function. Currently there is very little to offer the chronic headache patient. Nearly two thirds of patients discontinue prescription medications due to inadequate relief and side-effects. One target for pain management has been the cluster of nerve cells known as the sphenopalatine ganglion (SPG)—sometimes known as the pterygopalatine ganglion.

The SPG is the largest of the four parasympathetic ganglia associated with the trigeminal nerve and consists of the largest collection of neurons in the head outside of the brain. Early attempts to treat pain by targeting the SPG included injecting cocaine through the patient's face with a long needle. Contemporary pain treatments involve applying lidocaine to the SPG via a long cotton swab inserted through the nose, which allows the anesthetic to diffuse through the nasal cavity and block the SPG. Existing SPG block procedures involve great discomfort and risks for patients with the inconsistency of the medicine placement having an adverse impact on the results.

### SUMMARY

The invention provides a system and method for pain treatment that includes using a drug delivery device to deliver a medication that includes an antagonist of the calcitonin gene related peptide (CGRP). The drug delivery device delivers a medication that includes a CGRP agonist to provide an SPG block and gives patients immediate relief from headache pain with a simple, comfortable procedure. Systems of the invention include a drug delivery device configured for delivery of a medication that includes a CGRP agonist through the nasal cavity, effectively delivering an SPG block for migraine pain without the use of needles, cotton swabs, or harsh meds. The procedure is comfortable for patients and the drug delivery catheter allows a low risk, simple procedure for pain management. Systems and methods of the invention may be used to deliver active agents to medicate those aspects of the forebrain know to be involved with any number of pain conditions ranging from atypical and recurring headaches to various types of related pain disorders such as migraine or cluster headaches.

In certain aspects, the invention provides a drug delivery device for treating a pain condition. The device includes a catheter member defining an extended body with a proximal portion and a distal portion, wherein the distal portion defines a curved portion and a delivery port, a handle at the proximal portion of the catheter, and a first lumen and a second lumen within the catheter. Movement of an inner member results in delivery of a medication out of the delivery port at the distal portion of the catheter. The device includes a reservoir holding a medication that includes a CGRP receptor antagonist. The device may include one or more of a radiopaque marking band located at the distal portion of the catheter, a gripping surface with textured portions for grasping the device, or both. Preferably the proximal end of the catheter further comprises a visible marking on the handle that indicates a direction of travel of the medication and an orientation of the curved portion of the catheter member.

The device may include an arresting element on the handle, the arresting element comprising a groove and detent mechanism. In some embodiments, a portion of the resting element is pressed inward from the detent mechanism and slid in a proximal direction, causing the inner member (such as a plunger-type member or a pump type member) to increase pressure on the medication in the reservoir, thereby sending the medication through a lumen of the catheter such that the medication is delivered through the delivery port. The device may include means for delivery of agents effective for repolarization of cranial sensory and parasympathetic pathways via trigeminal primary afferent neurons.

Aspects of the invention provide a system for SPG neuro-modulation. The system includes a delivery device having a flexible catheter at least a portion of which is radiopaque, a hub and handle assembly at a proximal end of the flexible catheter, and a delivery mechanism within the hub and handle assembly operable to cause a medication to be delivered through the flexible catheter and out of a delivery port at a curved distal end of the catheter. The system includes a medication made available for delivery via the delivery device. The medication includes a CGRP antagonist such as telcagepant. The curved distal end of the catheter is configured for intranasal delivery of the mediation to the sphenopalatine ganglion (SPG). In some embodiments, the delivery mechanism comprises an inner plunger member operable to apply pressure to a fluid in the device operated by a trigger or slider on the hub and handle assembly. Preferably the system includes a three-dimensional indicia on the hub and handle assembly that shows an orientation of the curved distal end of the catheter.

In some aspects the invention provides a trans-nasal delivery system comprising a medication and a device configured for delivering the medication trans-nasally to a patient's SPG, the medication comprising a CGRP receptor antagonist.

### BRIEF DESCRIPTION OF THE FIGURES

FIG. 1 is a schematic showing a patient (normally supine) having an enhanced Sphenocath® brand of product emplaced within the patient's nasal anatomy;

FIG. 2 is a top side perspective view of an enhanced Sphenocath® brand of product;

FIG. 3 is a top view of an enhanced Sphenocath® brand of product;

FIG. 4 is a bottom view of an enhanced Sphenocath® brand of product;

FIG. 5 is a back-side bottom view of an enhanced Sphenocath® brand of product;

FIG. 6 is a front-side top view of an enhanced Sphenocath® brand of product;

FIG. 7 is a side view of an enhanced Sphenocath® brand of product; and,

FIG. 8 is an opposite-side view of an enhanced Sphenocath® brand of product.

FIG. 9 shows movement from a first to a second position of embodiments of the SphenoCath® brand of product.

FIG. 10 shows an exemplary chemical moiety which functions well for SPG circuit blockade.

### DETAILED DESCRIPTION

The invention provides a system and method for pain treatment that includes using a drug delivery device to deliver an SPG block that includes an agonist of the calcitonin gene related peptide (CGRP). CGRP is a member of the calcitonin family of peptides and is produced in both peripheral and central neurons. It is a potent peptide vasodilator and can function in the transmission of pain. CGRP is derived from dorsal root ganglion when synthesized in the dorsal horn of the spinal cord and may be linked to the transmission of pain. CGRP mediates its effects through a heteromeric receptor composed of a G protein-coupled receptor called calcitonin receptor-like receptor (CALCRL) and a receptor activity-modifying protein (RAMP1). Regulation of the calcitonin gene related peptide (CGRP) gene is in part controlled by the expression of the mitogen-activated protein kinases (MAPK) signaling pathway, cytokines such as TNFα and iNOS. 5HT1 receptor agonists, such as sumatriptan, increase intracellular calcium, which cause decreases in CGRP promoter activity. Receptor antagonists may also have the potential of limiting the effects of CGRP, noting that increased levels of CGRP have been reported in migraine disorders. Acute and chronic headaches have been classified according to various classification systems, calling various atypical headaches by labels from cluster headaches to trigeminal neuralgia, tension, or anything else modulated by the sphenopalatine ganglion (SPG). Such atypical headaches are marked by the need for immediate and accurate placement of medication to address them.

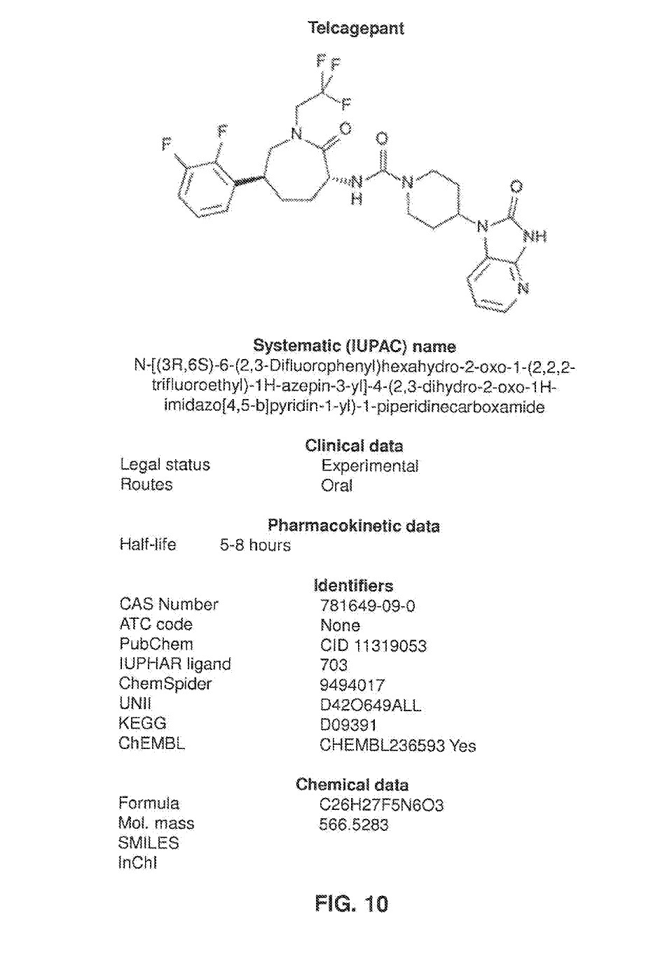

As detailed herein, “neuro-modulation” means a physiological response reflecting long-term changes persisting beyond the half-life of local anesthetics. The SPG refers to that ganglion—or set of nerves grouped together—that sends messages to other nerves, which is located in the back of the nasal cavity. “SPG Circuit Blockade” or SPG block includes delivery to elements of the SPG circuit of vehicles such as CGRP agonists, like that shown in FIG. 10. CGRP agonist includes compounds described in the literature or herein as antagonists of the CGRP.

The present inventor has discovered that dispersion of medication that includes a CGRP antagonist over the top of the middle turbinate provides for effective neuro-modulation. This is different from the temporary relief of a nerve block, because it can impact the multiple pathways connected to the SPG. By addressing placement and delivery of medication to the SPG, as opposed to quasi-random spraying of short-term agents, a new paradigm in pain relief has been entered. This standard of care shall likewise presently and directly be adopted and provides relief to patients, some of whom have suffered for years without relief.

Embodiments of the invention provide a drug delivery device that includes a medication, wherein the medication includes a CGRP receptor antagonist and optionally any suitable other ingredients. The invention also provides methods for pain relief that include using a drug delivery device to deliver medication that includes a CGRP receptor antagonist. Calcitonin gene-related peptide (CGRP) is a well-studied neuropeptide found at the very centers of the migraine processes. CGRP receptor antagonists disrupt the interaction of CGRP with its receptor and are being developed primarily for the acute treatment of migraine. Free CGRP and CGRP receptors can also be targeted using monoclonal antibodies which are being developed. Any suitable CGRP receptor antagonist may be used with devices, systems, and methods of the invention. For example, the CGRP receptor antagonist may include one of the CGRP receptor antagonists known as the ‘gepants’ that have been shown to have efficacy for the acute treatment of migraine.

One of the gepants—olcegepant (BIBN4096BS)—was discontinued because of difficulties in developing an oral formulation but may used with a delivery device of the invention (Olesen et al., 2004, Calcitonin gene related peptide receptor antagonist BIBN 4096 BS for the acute treatment of migraine. NEJM 350:1104-1110). Telcagepant (MK-0974) is a gepant that was discontinued because of concerns of liver toxicity after frequent use (Silberstein, 2013, Emerging target-based paradigms to prevent and treat migraine. Clin Pharmacol Ther 93:78-85; Hoffmann and Goadsby, 2012, New Agents for Acute Treatment of Migraine: CGRP Receptor Antagonists, iNOS Inhibitors. Curr Treat Options Neurol 14:50-59). MK-3207, a gepant molecule that was significantly more potent than telcagepant, was also discontinued because of concerns of liver toxicity (Salvatore et al., 2010, Pharmacological properties of MK-3207, a potent and orally active calcitonin gene-related peptide receptor antagonist. J Pharmacol Exp Ther 333:152-160; Pettypiece S., 2009, Merck Halts Testing of Migraine Drug on Liver Safety, Update2). The gepant BI44370A had efficacy demonstrated in a Phase 2 clinical study and may be delivered using devices and systems of the invention. (Diener et al., 2011, BI 44370 TA, an oral CGRP antagonist for the treatment of acute migraine attacks: Results from a phase II study. Cephalalgia 31:573-584). In addition to demonstrating proof of efficacy, CGRP receptor antagonist clinical trials demonstrated the tolerability of the class with acute dosing and that, as opposed to triptans, their use is not associated with vasoconstriction.

A fifth ‘gepant’ just joined the club by its own merits. In Cephalalgia 34(2):114-25 (2014), Marcus and colleagues report the result of a large Phase 2b study testing BMS-927711 for the acute treatment of migraine and that compound may be delivered using devices and systems of the invention. The primary efficacy endpoint was the proportion of pain-free subjects at 2 hours post dose. In addition to testing the primary endpoint for statistical significance compared with placebo, the authors attempted to define a measure of clinical relevance, or clinical response of at least 15% greater than the response of placebo. (Bigal, M., “BMS-927711 for the acute treatment of migraine” Cephalalgia 2014, Vol. 34 (2) pp. 90-92.)

The literature suggests that CGRP receptor antagonists are effective at multiple doses, meaning that all CGRP receptor antagonists tested to date have demonstrated efficacy in Phase 2 and Phase 3 studies.

Efficacy was established by the authors, although it was found to be numerically inferior to sumatriptan for this endpoint for all doses (although the study was not powered for direct comparisons). The drug also passed the bar of the secondary endpoints where most (and sometimes all) doses were superior to placebo, and most effective doses were also numerically superior to sumatriptan. Accordingly, certain doses of BMS-927711 seem to deliver levels of efficacy that are similar to those delivered by the highest dose of an effective triptan. Overall, the efficacy of the drug increased up to the dose of 75 mg when a plateau effect seemed to have been reached for doses between 75 mg and 300 mg. The highest tested dose (600 mg) demonstrated no additional benefit over the doses of 75 mg and 150 mg.

The most effective doses of the drug also significantly improved photophobia and phonophobia, which are known to be SPG-medicated results of many pain types.

The tolerability of BMS-927711 was reported as being placebo-like, which once more the authors conclude, reinforces the tolerability of the class for acute dosing. As with the other CGRP receptor antagonists, a pattern of side effects could not be identified and this is different from what is seen for other classes such as ergot derivatives and triptans (where certain adverse events, such as chest tenderness and muscle tightness seem to be class-specific). No serious adverse event was reported in the trial.

In sum, the efficacy of a single dose of BMS-927711 has been demonstrated. In this regard, one would expect Phase 3 to focus on defining consistency of efficacy when treating multiple attacks. Tolerability also seems to be consistent with expectations for the class, and one would expect Phase 3 to reinforce the finding. Accordingly, Phase 3 would be mainly about defining the safety of the drug, especially its effect on the liver in situations of frequent dosing and in patients using medications that are metabolized by the CYP3A4. A drug that has sumatriptan-like efficacy, that may be better tolerated, and without vasoconstrictive properties, would be an incredible addition to the migraine treatment arsenal, clearly addressing current unmet needs. In particular, patients with existing vascular disease or vascular risk factors currently have only very limited choices of acute migraine medications.

Any other suitable CGRP receptor antagonist may be used in systems and methods of the invention. For example, N-[1-(2,3-Difluorobenzyl)-5-oxo-4-(2,22-trifluoroethyl)-1,4-diazepan-6-yl]-4-(2-oxo-2,3-dihydro-1H-imidazo[4,5-b]pyridin-1-yl)piperidine-1-carboxamide may be used. Other CGRP receptor antagonists suitable for use with the invention include those discussed in U.S. Pat. No. 7,772,224 to Paone; U.S. Pub. 2005/0215576 to Degnan; U.S. Pub. 2007/0148093 to Conway; U.S. Pat. No. 7,220,862 to Chaturvedula; and U.S. Pub. 2004/0063735, the contents of each of which are incorporated by reference for all purposes.

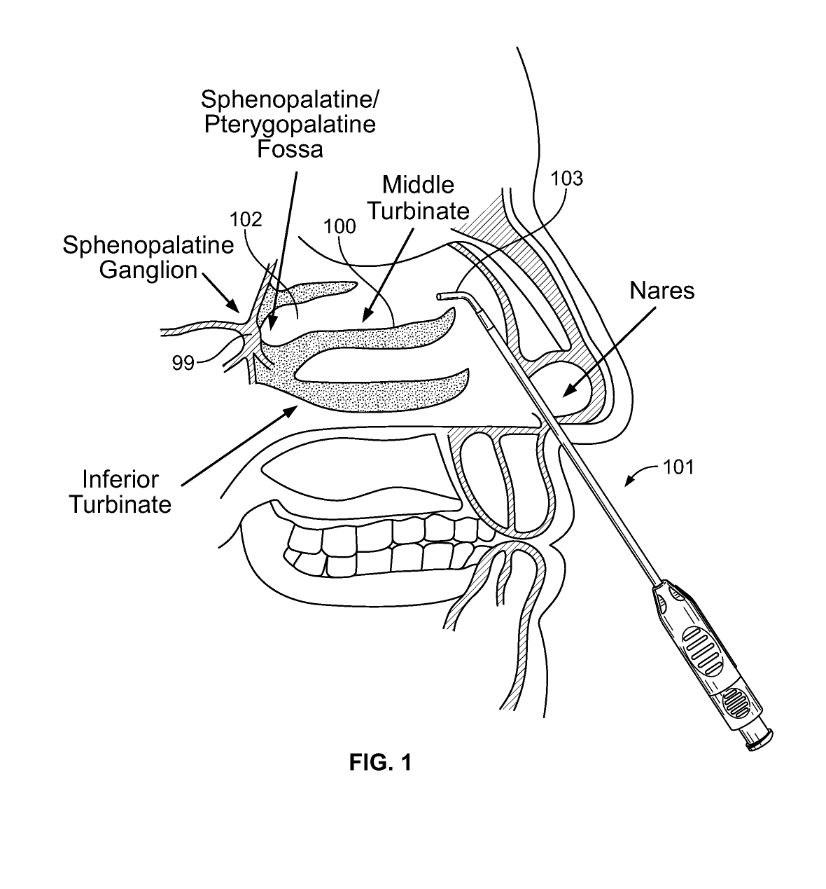

Referring now to FIG. 1, it is seen that SPG 99 functions as a biologic “circuit” along with other neuronal cell bodies, including but not limited to those autonomic ganglia known as the Stellate Lumbar Sympathetic, Celiac Plexus (and others). By directly contacting and medicating SPG 99, a collective chemical repolarization of these autonomic ganglia is achieved. Physiologically, this type of collective chemical repolarization is analogous to the type of reset mechanism used to counter atrial fibrillation, when the heart undergoes electrical repolarization in cardioversion.

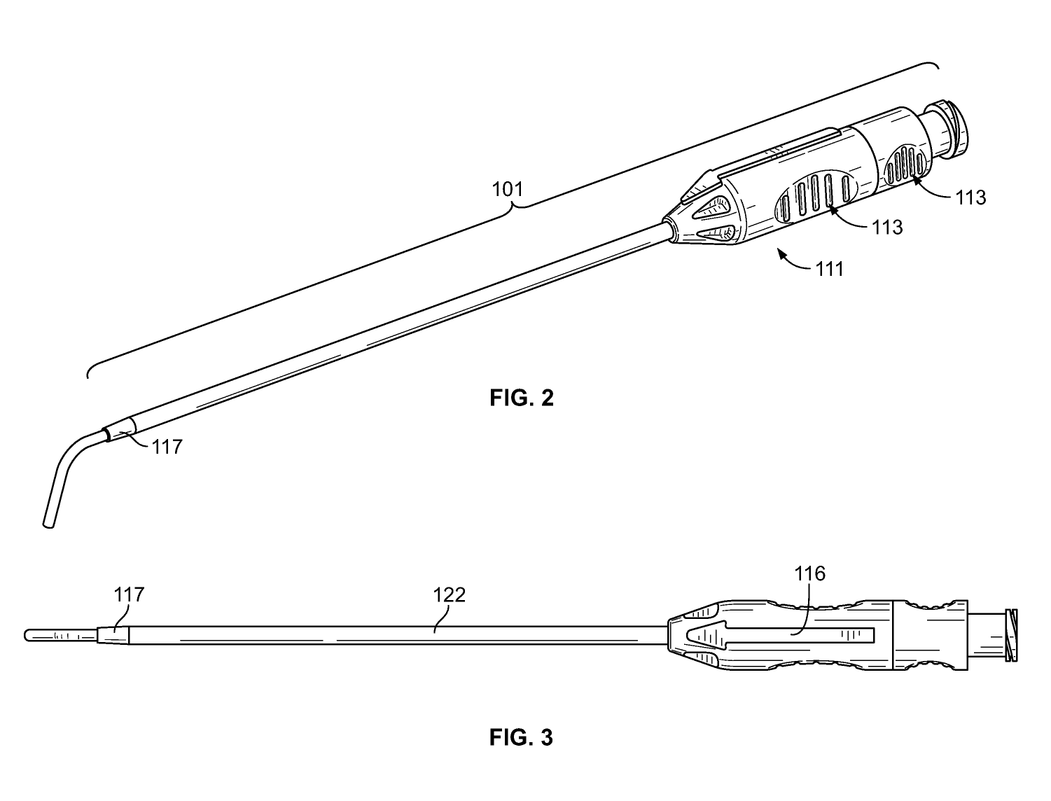

FIG. 2 shows improved drug delivery catheter 101, including ergonomic hub/handle 111, feature finger gripping means 113 placed on the side of device 101. The drug delivery catheter 101 may be the drug delivery catheter sold under the trademark SPHENOCATH by Dolor Technologies, LLC (Riverton, Utah). The drug delivery catheter 101 may be the drug delivery catheter as shown in U.S. Pat. No. 8,388,600 to Eldredge, the contents of which are incorporated by reference in their entirety for all purposes.

FIG. 3 shows directional arrow 116 and radiopaque marking tip 117, which is often made of tungsten (W). Those skilled in the art understand tungsten, tantalum and/or platinum may further be disposed within shaft 122 of device 101 for visualization under imaging technologies.

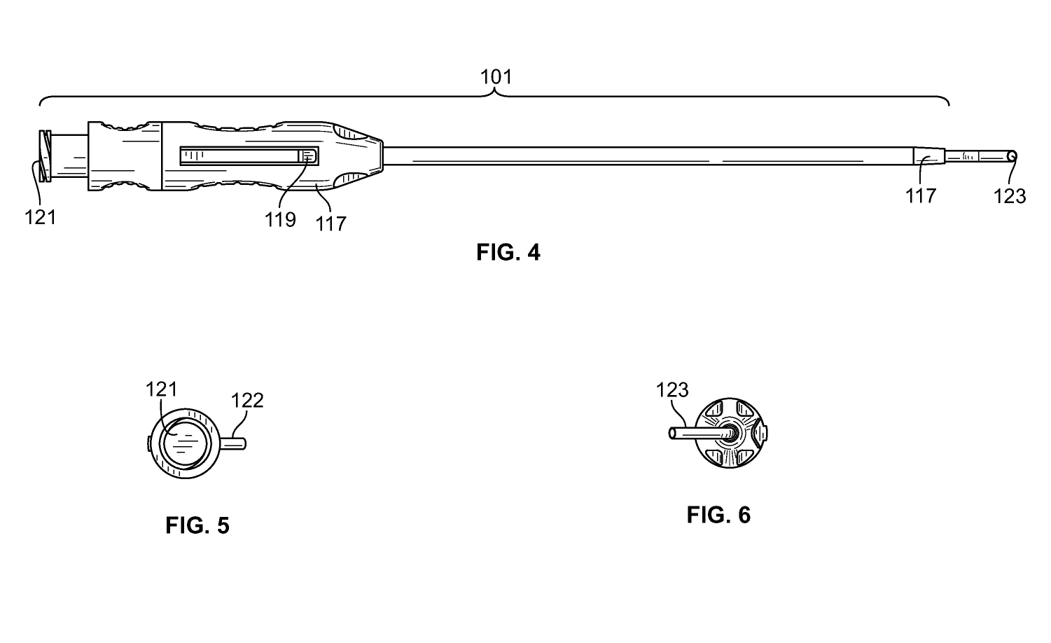

FIG. 4 is a ventral view, showing device 101 with its ergonomic hub/handle 111, radiopaque marker 117, and grooved arresting element 119. Infusion port 121 and medication delivery port 123 demonstrate a relationship between deflecting tip angle for delivery port 123.

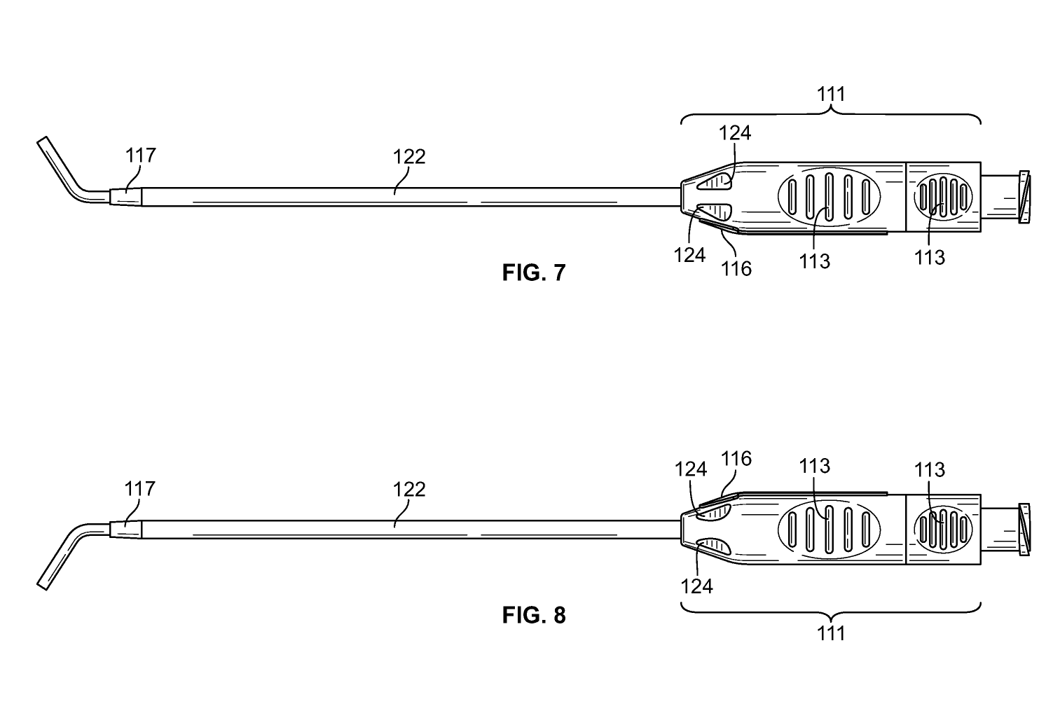

FIG. 7 and FIG. 8, each side views, demonstrate ergonomic hub/handle 111 with further gripping features 124 and the relationship between directional arrow 116 and deflecting tip/medication delivery port 123—namely, arrow 116 shows orientation of deflecting tip/medication delivery port 123 which angles shown by arrow 138 allows deflecting tip 123 to bend in the direction of a patient's SPG to deliver, for example, CGRP and the like actives and medications.

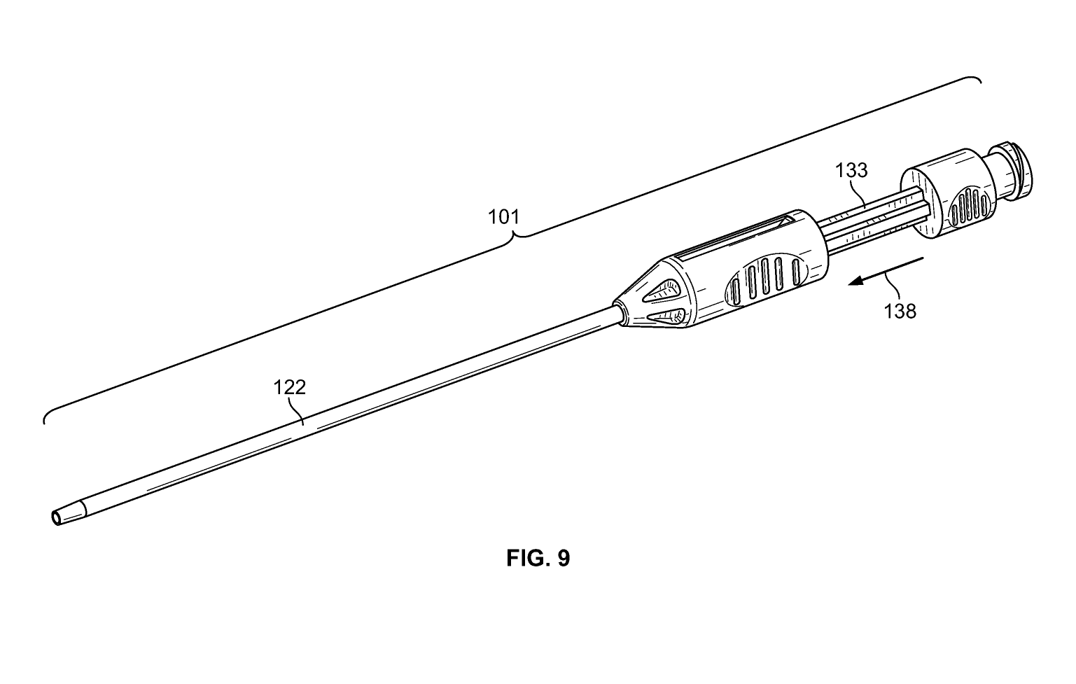

Referring still to FIG. 1 and FIG. 9, the drug delivery device 101 is emplaced while patient is supine, and extends in a first position, past the middle turbinate 100 to access the pterygopalatine/sphenopalatine fossa 102, which is adjacent to the SPG 99. As shown in FIG. 9, direction 138 shows how medication travels down the path as the catheter moves from a first position to a second position, where stiffening member 133 is extended.

FIG. 10 shows the type of agent which works as a CGRP receptor antagonist with the instant system. Namely, this moiety, or others, when directly delivered to the SPG circuit, functions as CGRP receptor antagonist.

FIGS. 4-10 show a drug delivery device 101 for treating a pain condition. The device 101 includes a catheter member 122 defining an extended body with a proximal portion and a distal portion, wherein the distal portion defines a curved portion and a delivery port 123, a handle 111 at the proximal portion of the catheter, and a first lumen and a second lumen within the catheter. Movement of an inner member allows egress out of the delivery port at the distal portion of the catheter. The device 101 includes reservoir within the device holding a medication that includes a calcitonin gene-related peptide (CGRP) receptor antagonist. The device 101 may include one or more of a radiopaque marking band 117 located at the distal portion of the catheter, a gripping surface with textured portions 113 for grasping the device, or both. Preferably the proximal end of the catheter further comprises a visible marking 116 on the handle that indicates a direction of travel of the medication and an orientation of the curved portion of the catheter member.

The device 101 may include an arresting element 119 on the handle, the arresting element comprising a groove and detent mechanism. The device may include means for delivery of agents effective for repolarization of cranial sensory and parasympathetic pathways via trigeminal primary afferent neurons.

Aspects of the invention provide a system for SPG neuro-modulation. The system includes a delivery device 101 having a flexible catheter at least a portion of which is radiopaque, a hub and handle assembly at a proximal end of the flexible catheter, and a delivery mechanism (not pictured) within the hub and handle assembly operable to cause a medication to be delivered through the flexible catheter 122 and out of a delivery port 123 at a curved distal end of the catheter. The system includes a medication made available for delivery via the delivery device 101, the medication comprising a CGRP antagonist such as telcagepant. The curved distal end of the catheter is configured for intranasal delivery of the mediation to the sphenopalatine ganglion (SPG). In some embodiments, the delivery mechanism comprises a plunger member operable to apply pressure to a fluid in the device. Preferably the system includes a three-dimensional indicia on the hub and handle assembly that shows an orientation of the curved distal end of the catheter.

In some aspects the invention provides a trans-nasal delivery system comprising a medication and a device configured for delivering the medication trans-nasally to the SPG, the medication comprising a CGRP receptor antagonist.

While methods, devices, compositions, and the like, have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims. It is understood that the term, present disclosure, in the context of a description of a component, characteristic, or step, of one particular embodiment of the disclosure, does not imply or mean that all embodiments of the disclosure comprise that particular component, characteristic, or step.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

This application expressly incorporates by reference U.S. Letters Pat. No. 8,388,600; International application PCT/US2012/027138 (published as WO2013/119258); European Patent Application no. 12867745.7; and U.S. patent applications Ser. Nos. 12/553,953; 13/371,288; and 13/629,992; as if each was fully set forth herein, in their entirety.

1. A drug delivery device for treating a pain condition, the device comprising:
a catheter member defining an extended body with a proximal portion and a distal portion, wherein the distal portion defines a curved portion and a delivery port;
a handle at the proximal portion of the catheter;
a first lumen and a second lumen in the catheter, wherein movement of an inner member allows egress out of the delivery port at the distal portion of the catheter; and
a reservoir within the device holding a medication that includes a calcitonin gene-related peptide (CGRP) receptor antagonist.

2. The drug delivery device of claim 1, further comprising at least a radiopaque marking band located at the distal portion of the catheter.

3. The drug delivery device of claim 1, wherein the handle further comprises a gripping surface with textured portions for grasping the device.

4. The drug delivery device of claim 1, wherein said proximal end of the catheter further comprises a visible marking on the handle indicating:
a direction of travel of the medication, and
an orientation of the curved portion of the catheter member.

5. The drug delivery device of claim 1, wherein said proximal end of the catheter further comprises a three-dimensional marking on the handle that indicates a direction of travel of the medication and an orientation of the curved portion of the catheter member.

6. The drug delivery device of claim 5, wherein the three-dimensional marking comprises a raised and curved area on the handle defining a curve that mimics the curved portion of the catheter such that an operator can determine an orientation of the catheter member via tactile stimulus from the three-dimensional marking.

7. The drug delivery device of claim 1, further comprising an arresting element on the handle, the arresting element comprising a groove and detent mechanism.

8. The drug delivery device of claim 1, further comprising means for delivery of agents effective for repolarization of cranial sensory and parasympathetic pathways via trigeminal primary afferent neurons.

9. A system for SPG neuro-modulation, the system comprising:
a delivery device comprising
a flexible catheter at least a portion of which is radiopaque,
a hub and handle assembly at a proximal end of the flexible catheter, and
a delivery mechanism operable at the hub and handle assemble to cause a medication to be delivered through the flexible catheter and out of a delivery port at a curved distal end of the catheter; and
a medication made available for delivery via the delivery device, the medication comprising a CGRP antagonist.

10. The system of claim 9, wherein the CGRP antagonist is telcagepant.

11. The system of claim 9, wherein the delivery mechanism comprises a plunger member operable to apply pressure to a fluid in the device.

12. The system of claim 9, further comprising a three-dimensional indicia on the hub and handle assembly, the three-dimensional indicia showing an orientation of the curved distal end of the catheter.

13. The system of claim 12, wherein the three-dimensional indicia is configured to be detected via touch by a hand of an operator to indicate the orientation of the curved distal end of the catheter.

14. The system of claim 9, wherein the curved distal end of the catheter is configured for intranasal delivery of the medication to the sphenopalatine ganglion (SPG).

15. The system of claim 14, wherein the CGRP antagonist is BMS-927711.

16. A trans-nasal delivery system comprising a medication and a device configured for delivering the medication trans-nasally to the SPG, the medication comprising a CGRP receptor antagonist.

17. The delivery system of claim 16, wherein the device comprises a catheter with a curved distal end configured for intranasal delivery.

18. The delivery system of claim 17, wherein the device comprises a handle at a proximal end of the catheter and the handle comprises a marker feature that is shaped to represent the curved distal end of the catheter and is detectable by touch of a user to inform the user of an orientation of the curved distal end.

19. The delivery system of claim 18, wherein the CGRP receptor antagonist is BMS-927711.
